(19) United States
(12) Patent Application Publication
(10) Pub. No.: US 2019/0262159 A1
(43) Pub. Date: Aug. 29, 2019

(54) ANKLE BRACE FOR PREVENTING OR TREATING HIGH ANKLE SPRAINS

(71) Applicant: Christopher James Curran, Novi, MI (US)

(72) Inventor: Christopher James Curran, Novi, MI (US)

(21) Appl. No.: 16/283,763

(22) Filed: Feb. 23, 2019

Related U.S. Application Data
(60) Provisional application No. 62/634,269, filed on Feb. 23, 2018.

(51) Int. Cl. A61F 5/01; A61F 5/30

(52) U.S. Cl. CPC A61F 5/0111; A61F 13/067; A61F 5/30; A61F 5/0127

(57) ABSTRACT

An ankle brace according to the present disclosure includes a medial pad, a lateral pad, a posterior wall, and at least one horizontal strap. The medial pad is configured to engage a medial side of an ankle. The lateral pad is configured to engage a lateral side of the ankle. The posterior wall is attached to the medial pad and is configured to engage a posterior side of the ankle. The at least one horizontal strap is configured to be secured to the lateral pad, secured to the posterior wall, and pulled in an anterior direction to apply an anterior force to a distal fibula of the ankle. The anterior force brings the distal fibula closer to a distal tibia of the ankle and thereby decreases stress on ligaments connecting the distal fibula and the distal tibia to one another.

### CROSS-REFERENCE TO RELATED APPLICATIONS

This application claims the benefit of U.S. Provisional Application No. 62/634,269, filed on Feb. 23, 2018. The entire disclosure of the application referenced above is incorporated herein by reference.

### FIELD

The present disclosure relates to ankle braces configured to prevent or treat high ankle sprains.

### BACKGROUND

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Ankle braces are typically designed to prevent inversion and eversion sprains of the lower ankle. Many ankle braces will have medial and lateral pads, supports, and straps secured against the lower leg. The purpose of these is often to provide support to the medial and lateral sides of the ankle to help prevent or treat inversion or eversion ankle sprains by limiting inversion and eversion of the ankle. These braces are not designed to compress the syndesmosis joint between the distal tibia and fibula or to migrate the distal fibula anteriorly so to bring the distal fibula closer to the distal tibia. To this end, these braces typically include a rigid heelpiece that extends around the heel and connects the medial and lateral pads to one another while inhibiting movement of the pads in the medial-lateral direction, and thereby inhibiting compression of the syndesmosis joint between the distal tibia and fibula. In addition, these braces are not designed to support the ligaments between the distal tibia and fibula that are often injured in a high ankle sprain. The term high ankle sprain is used because it is an injury to the lower leg and ankle that is superior to the typical inversion or eversion lower ankle sprain.

The ligaments of the lower leg between the distal tibia and distal fibula that are injured in a high ankle sprain include the interosseous membrane, the anterior inferior tibiofibular ligament, and the posterior inferior tibiofibular ligament. These ligaments can be injured when the foot is dorsiflexed and externally rotated with extreme forces such as in athletic injuries. This position and motion of the foot and ankle stresses the space between the tibia and fibula. This increased tension stresses the ligaments holding these bones together.

### SUMMARY

A first example of an ankle brace according to the present disclosure includes a medial pad, a lateral pad, a posterior wall, and at least one horizontal strap. The medial pad is configured to engage a medial side of an ankle. The lateral pad is configured to engage a lateral side of the ankle. The posterior wall is attached to the medial pad and is configured to engage a posterior side of the ankle. The at least one horizontal strap is configured to be secured to the lateral pad, secured to the posterior wall, and pulled in an anterior direction to apply an anterior force to a distal fibula of the ankle. The anterior force brings the distal fibula closer to a distal tibia of the ankle and thereby decreases stress on ligaments connecting the distal fibula and the distal tibia to one another.

In one example, the ankle brace further includes at least one ring attached to a lateral edge of the posterior wall, and the at least one horizontal strap is configured to be inserted through the at least one ring to secure the at least one horizontal strap to the posterior wall.

In one example, the ankle brace further includes an anterior wall attached to the medial pad and configured to engage an anterior side of the ankle, and the at least one horizontal strap is attached to the anterior wall and is configured to be inserted through slits in the lateral pad, through the at least one ring, and fastened to the anterior wall to secure the at least one horizontal strap to the lateral pad.

In one example, the at least one horizontal strap includes a first portion attached to the anterior wall and having a plurality of loops, and a second portion having a plurality of hooks configured to engage the plurality of loops and thereby fasten the at least one horizontal strap to the anterior wall.

In one example, the lateral pad includes an inner liner and an outer cover that is attached to the inner liner, wherein the outer cover defines the slits in the lateral pad.

In one example, the anterior and posterior walls are formed by a flexible sheet, and the flexible sheet includes a medial portion that forms an inner liner of the medial pad.

In one example, the medial pad includes the medial portion of the flexible sheet, a medial insert, and an outer cover attached to the medial portion of the flexible sheet to form a pocket that encloses the medial insert.

A second example of an ankle brace according to the present disclosure includes a medial pad, a lateral pad, a vertical strap, and at least one horizontal strap. The medial pad is configured to engage a medial side of an ankle. The lateral pad is configured to engage a lateral side of the ankle. The vertical strap is attached to one of the medial and lateral pads and configured to be wrapped around a heel and secured to the other one of the medial and lateral pads to prevent the ankle brace from moving in a superior direction. The at least one horizontal strap secures the medial and lateral pads to one another and is configured to be pulled in at least one of a medial direction and a lateral direction to move the medial and lateral pads toward one another such that the medial and lateral pads apply a compression force to the ankle in the medial and lateral directions. The compression force urges a distal fibula of the ankle and a distal tibia of the ankle toward one another. The vertical strap is flexible so that the vertical strap does not counteract the compression force applied by the medial and lateral pads.

In one example, the vertical strap is attached to the medial pad and is configured to be wrapped around the heel and secured to the lateral pad.

In one example, the lateral pad includes a plurality of loops and the vertical strap includes a plurality of hooks configured to engage the plurality of loops to secure the vertical strap to the lateral pad.

In one example, the at least one horizontal strap is configured to be pulled in the lateral direction to apply the compression force to the ankle.

In one example, the ankle brace further includes a posterior wall and at least one ring, the posterior wall is attached to the medial pad and is configured to engage a posterior side of the ankle, the at least one ring is attached to a lateral edge of the posterior wall, and the at least one horizontal strap is configured to be inserted through the at least one ring to secure the at least one horizontal strap to the posterior wall.

In one example, at least a portion of the vertical strap is made of neoprene rubber having a thickness between 2 millimeters and 7 millimeters.

In one example, the medial pad has a first stiffness and the lateral pad has a second stiffness that is greater than the first stiffness.

A third example of an ankle brace according to the present disclosure includes a medial pad, a lateral pad, and at least one horizontal strap. The medial pad is configured to engage a medial side of an ankle. The medial pad has a first width adjacent to its superior end and a second width adjacent to its inferior end that is greater than the first width. The lateral pad is configured to engage a lateral side of the ankle. The lateral pad has a third width adjacent to its superior end and a fourth width adjacent to its inferior end that is greater than the first width. The at least one horizontal strap secures the medial and lateral pads to one another and is configured to be pulled in at least one of a medial direction and a lateral direction to move the medial and lateral pads toward one another such that the medial and lateral pads apply a compression force to the ankle in the medial and lateral directions. The compression force urges a distal fibula of the ankle and a distal tibia of the ankle toward one another.

In one example, the second width is at least 1.25 times greater than the first width, and the fourth width is at least 1.25 times greater than the third width.

In one example, the lateral pad includes an inner liner and an outer cover that is attached to the inner liner.

In one example, each of the inner liner and the outer cover is made from neoprene rubber having a thickness between 2 millimeters and 7 millimeters.

In one example, the medial pad includes an inner liner, a medial insert, and an outer cover attached to the inner liner to form a pocket that encloses the medial insert.

In one example, each of the inner liner and the outer cover is made from neoprene rubber having a thickness between 2 millimeters and 7 millimeters, and the medial insert is made from high-density foam having a thickness between 2 millimeters and 10 millimeters.

Further areas of applicability of the present disclosure will become apparent from the detailed description, the claims and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIG. 1 is a medial view of an example of a right ankle brace according to the principles of the present disclosure;

FIG. 2 is a lateral view of the right ankle brace of FIG. 1;

FIG. 3 is an anterior view of the right ankle brace of FIG. 1;

FIG. 4 is a posterior view of the right ankle brace of FIG. 1;

FIG. 5 is a perspective view of a stabilizer body included in the right ankle brace of FIG. 1;

FIG. 6 is a perspective view of a stabilizing insert included in the right ankle brace of FIG. 1;

FIG. 7 is a perspective view of a medial pad outer cover included in the right ankle brace of FIG. 1;

FIG. 8 is a perspective view of a vertical heel stirrup strap included in the right ankle brace of FIG. 1;

FIG. 9 is a perspective view of look locks and a hook securing strap included in the right ankle brace of FIG. 1;

FIG. 10 is a planar view of a horizontal loop strap included in the right ankle brace of FIG. 1;

FIG. 11 is a perspective view of a lateral pad outer cover included in the right ankle brace of FIG. 1;

FIG. 12 is a perspective view of a lateral pad inner liner included in the right ankle brace of FIG. 1;

FIG. 13 is a medial view of the right ankle brace of FIG. 1 secured to the ankle and foot of a person;

FIG. 14 is a posterior view of the right ankle brace of FIG. 1 secured to the ankle and foot;

FIG. 15 is a lateral view of the right ankle brace of FIG. 1 secured to the ankle and foot; and

FIG. 16 is an anterior view of the right ankle brace of FIG. 1 secured to the ankle and foot.

In the drawings, reference numbers may be reused to identify similar and/or identical elements.

### DETAILED DESCRIPTION

An ankle brace according to the present disclosure includes medial and lateral pads that act like a vice when pulled together by horizontal loop straps to apply a compression force to the distal fibula and distal tibia to stabilize the syndesmosis joint between these bones. Due to the direction that the horizontal loop straps are secured, the medial and lateral pads also act to pull the distal fibula anteriorly and medially to bring the distal fibula closer to the distal tibia. These two actions (i.e., the compression force and the anterior-medial force) bring the distal fibula and distal tibia closer together and can decrease stress on the ligaments between these bony structures, which include the interosseous membrane, anterior inferior tibiofibular ligament, and the posterior inferior tibiofibular ligament.

The ability of the brace to apply the compression force is enabled at least in part by the thickness and material of the medial and lateral pads, the shape of the pads, and the fact that the horizontal loop straps are pulled laterally to secure the brace to the ankle. In addition, the brace includes a vertical heel strap that prevents the brace from migrating superiorly, and the vertical heel strap is flexible so that it does not counteract the compression force. The ability of the brace to apply the anterior-medial force is enabled at least in part by the fact that the brace includes a posterior wall and rings attached to the lateral edge of the posterior wall, as well as the shape of the lateral pad. The posterior wall engages the distal fibula as the horizontal loop straps are pulled anteriorly. The horizontal loop straps are inserted laterally through the rings before being pulled anteriorly. Regarding the shape of the pads, the pads are wider at their inferior ends to match the shape of the distal fibula and the distal tibia to maximize the contact between the pads and the corresponding bone when the two bones are brought closer together.

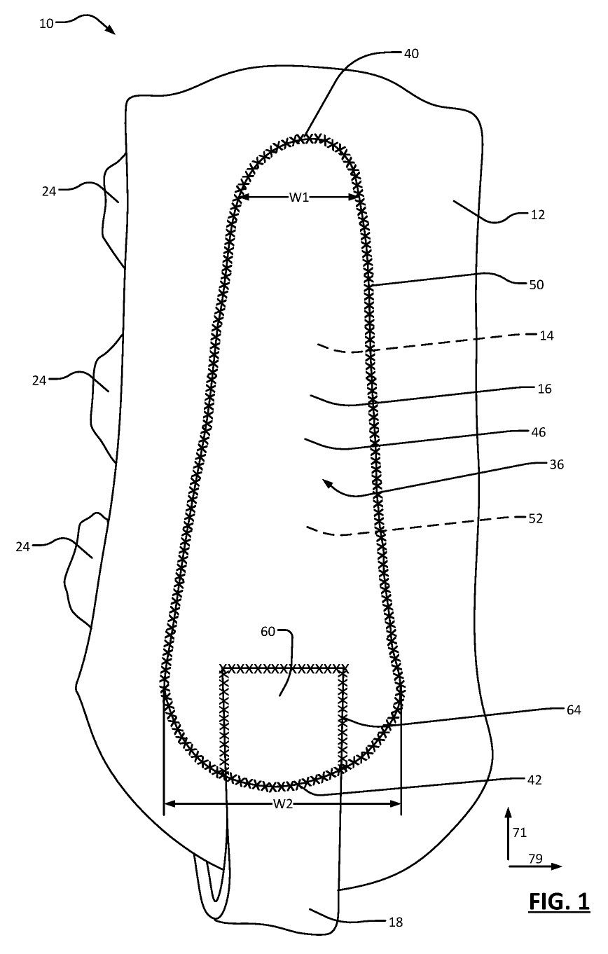

Referring now to FIGS. 1-4, an ankle brace 10 is specifically made for either a left foot and ankle or a right foot and ankle. The brace 10 includes a main or stabilizer body 12, a medial stabilizing insert 14, a medial pad outer cover 16, a vertical heel stirrup strap 18, hook tabs 20, rings or loop locks 22, hook securing straps 24, horizontal loop straps 26, a lateral pad inner liner 28, and a lateral pad outer cover 30.

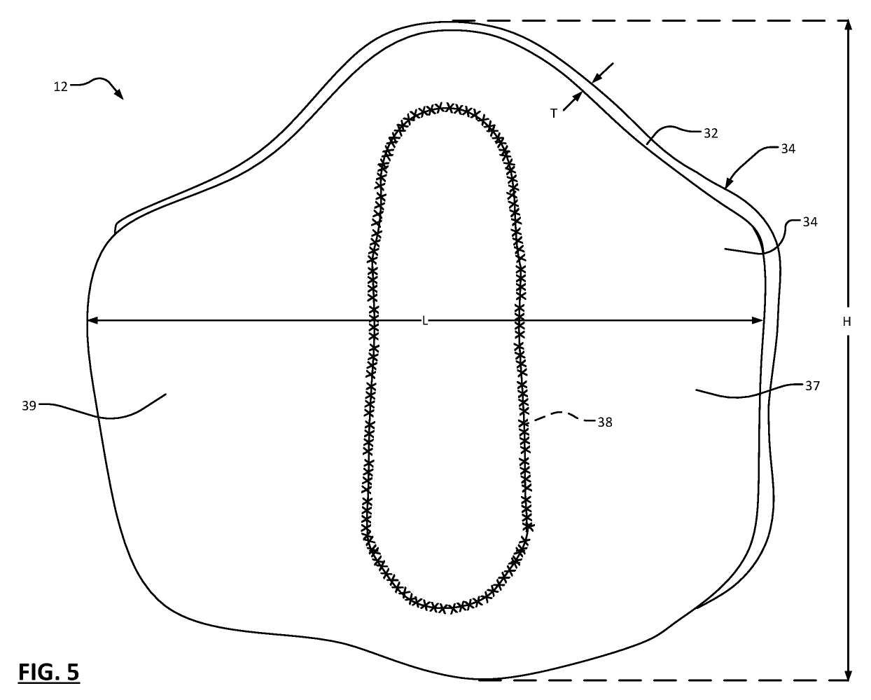

Referring now to FIG. 5, the stabilizer body 12 is made of a flexible sheet of material (e.g., a wrap). In one example, the stabilizer body 12 is made from a neoprene rubber 32 with a nylon laminate 34 on both sides. The stabilizer body 12 includes a medial portion 38 that forms part of a medial pad 36 (FIGS. 1, 3, and 4), as discussed below. In addition, the stabilizer body 12 forms an anterior wall 37 of the brace 10 and a posterior wall 39 of the brace 10. The anterior wall 37 is configured to engage the anterior side of an ankle. The posterior wall 39 is configured to engage the posterior side of an ankle. Since the medial portion 38 of the stabilizer body 12, the anterior wall 37, and the posterior wall 39 are integrally formed as a unitary body (i.e., the stabilizer body 12), the anterior wall 37 and the posterior wall 39 are each attached to the medial pad 36.

The stabilizer body 12 can have a maximum length L between 7 inches and 11 inches, a maximum height H between 6.5 inches and 10.5 inches, and/or a thickness T between 2 millimeters (mm) and 7 mm. The material of the stabilizer body 12 may vary and can include nylon, cotton, stretchable fabric, non-stretchable fabric, or any suitable material. The shape, quantity, length, width, height, thickness, breathability, ability to stretch, firmness, and color of the stabilizer body 12 may all vary.

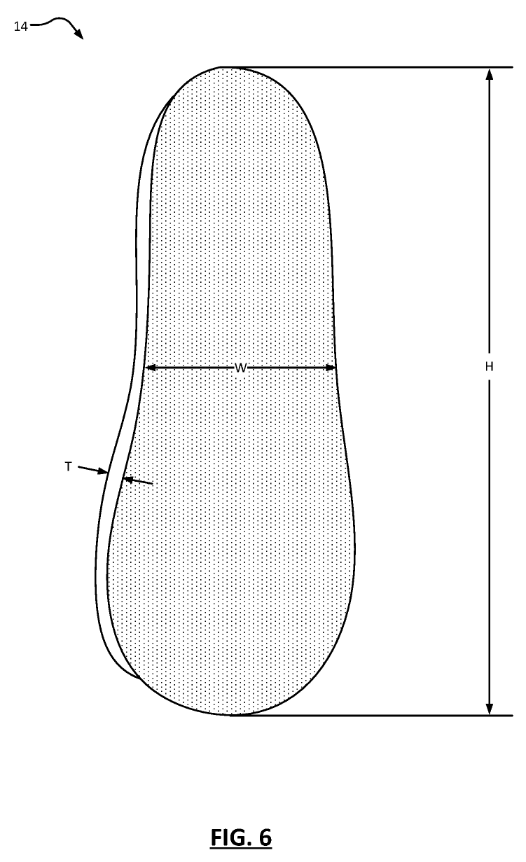

Referring now to FIG. 6, the stabilizing insert 14 can be teardrop-shaped as shown and can be made from a high-density foam. The stabilizing insert 14 can have a thickness T between 2 mm and 10 mm (e.g., a thickness between 4.5 mm and 8.5 mm), a height H between 5 inches and 9 inches, and/or a width W between 1 inch and 3 inches. The stabilizing insert 14 may be comprised of foam rubber, moldable or non-moldable plastic, metal or other suitable materials, and may be any combination of these materials. In various implementations, the insert 14 may be omitted from the brace 10. The material, quantity, shape, length, width, height, thickness, breathability, ability to stretch, firmness, and color of the stabilizing insert 14 may vary. The stabilizing insert 14 may or may not be secured to the stabilizer body 12.

Referring now to FIGS. 1-5, the medial pad 36 of the ankle brace 10 consists of the medial portion 38 (FIG. 5) of the stabilizer body 12, the stabilizing insert 14, and the medial pad outer cover 16. The medial portion 38 of the stabilizer body 12 acts as an inner liner for the medial pad 36. The medial pad 36 is configured to engage the medial side of an ankle. The medial pad 36 may have a first width W1 adjacent to its superior end 40 and a second width W2 adjacent to its inferior end 42. The second width W2 of the medial pad 36 may be greater than the first width W1 of the medial pad 36. In one example, the second width W2 of the medial pad 36 is at least 1.25 times greater than the first width W1 of the medial pad 36. The medial pad 36 can be positioned with its wider end (i.e., its inferior end 42) distally against the medial malleolus of the tibia and extending proximally up the distal tibia.

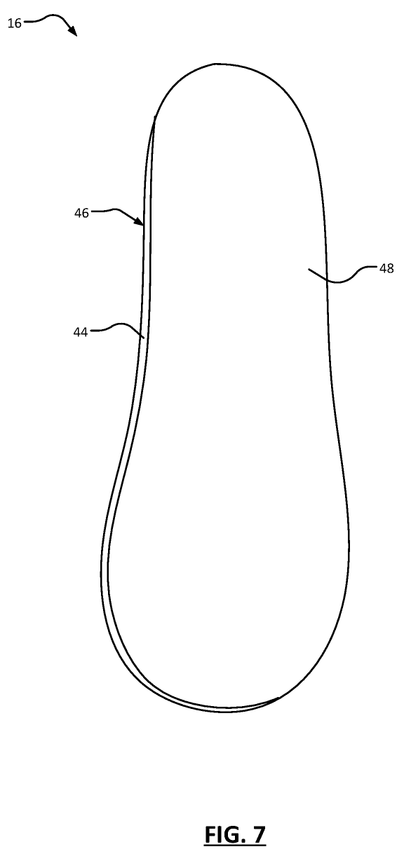

With additional reference to FIG. 7, the medial pad outer cover 16 can be teardrop-shaped as shown and can be made from a neoprene rubber 44 with a hook-compatible laminate 46 on the outside and a non-stretch laminate 48 on the inside. As used herein, “hook-compatible laminate” may refer to laminate having a plurality of loops configured to interlock with a plurality of hooks such as those found in hook-and-loop fasteners. The medial pad outer cover 16 can have a height H between 5 inches and 10 inches, a width W between 1 inch and 4 inches, and/or a thickness T between 2 mm and 7 mm. The exterior surface of the medial pad outer cover 16 is the side with the hook-compatible laminate 46. The material, quantity, shape, length, width, height, thickness, breathability, ability to stretch, firmness, and color of the medial pad outer cover 16 may vary. In addition, the method of attaching the medial pad outer cover 16 to the stabilizer body 12 may vary. Further, the medial pad outer cover 16 may or may not be attached to the stabilizer body 12.

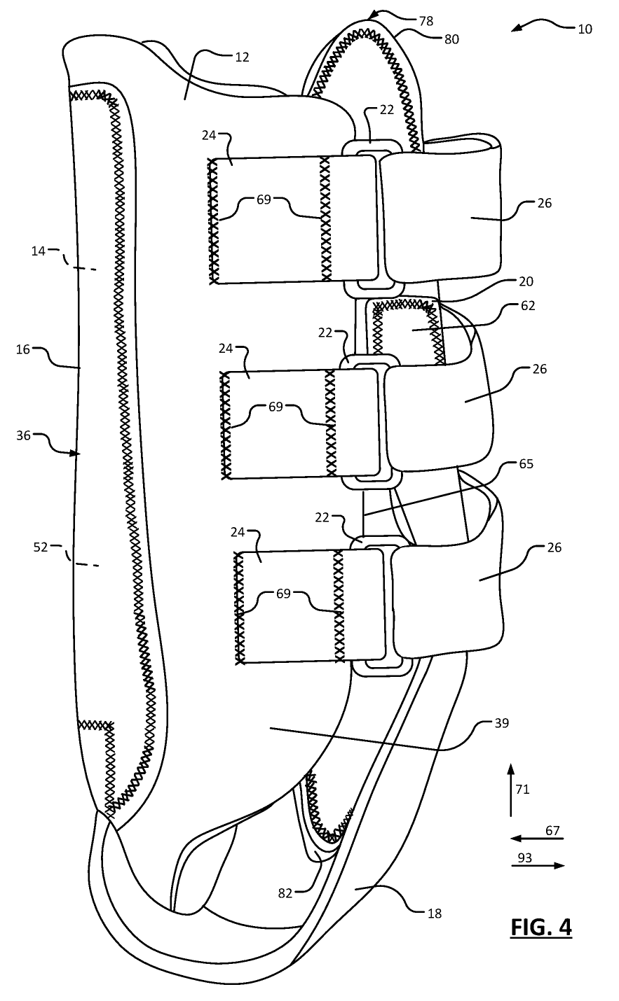

As best shown in FIGS. 1 and 4, the medial pad outer cover 16 is joined (e.g., sewn) to the exterior of the medial portion 38 of the stabilizer body 12 (e.g., via stiches 50) to form a pocket 52. The stabilizing insert 14 is positioned between the stabilizer body 12 and the medial pad outer cover 16 and is enclosed within the pocket 52 formed by joining the medial pad outer cover 16 to the medial portion 38 of the stabilizer body 12. The firmness of the stabilizing insert 14 gives the brace 10 rigidity against the tibia without causing discomfort when the brace 10 compresses the tibia and fibula together and not allowing the stabilizer body 12 to over-stretch and lose its compressive forces.

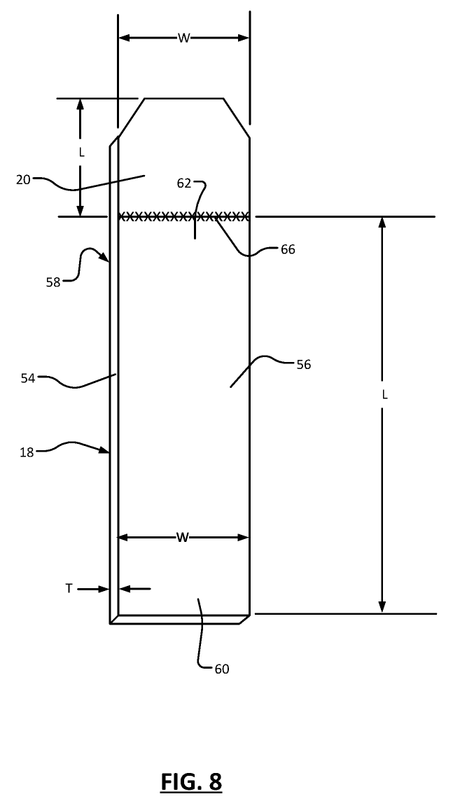

Referring now to FIGS. 1-4 and 8, the vertical heel stirrup strap 18 can be made of a neoprene rubber 54 with a hook-compatible laminate 56 on the outer side and smooth neoprene skin 58 on the underside. The vertical heel stirrup strap 18 has a first end 60 and a second end 62 opposite of the first end 60. The first end 60 of the vertical heel stirrup strap 18 is attached (e.g., sewn) to the stabilizer body 12 of the brace 10 at the exterior and bottom of the medial pad outer cover 16 (e.g., using stitches 64). The second end 62 of the vertical heel stirrup strap 18 has a hook tab 20 attached (e.g., sewn) thereto (e.g., using stitches 66). The hook tab 20 may be considered part of the vertical heel stirrup strap 18. The vertical heel stirrup strap 18 can have a length L between 5 inches and 12 inches, a width W between 1 inch and 3 inches, and/or a thickness T between 2 mm and 7 mm. The materials, quantity, shape, length, width, height, thickness, breathability, ability to stretch, firmness, and color of the vertical heel stirrup strap 18 may vary.

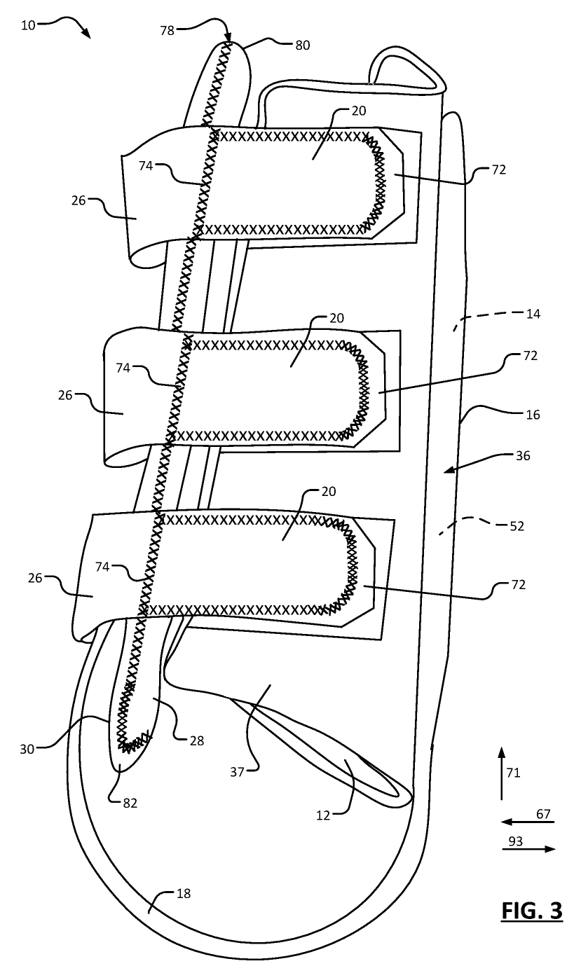

The vertical heel stirrup strap 18 extends from the inferior medial side of the brace 10 down vertically to the bottom and then up the lateral side of the brace 10. While applying the brace 10 to the ankle and lower leg, the athlete gently pulls the vertical heel stirrup strap 18 below their heel, then in a lateral direction 67 (FIG. 3) of the brace 10, and attaches it with the hook tab 20 along the lateral pad outer cover 30. When applied, the vertical heel stirrup strap 18 acts to secure the brace 10 to the ankle and lower leg and prevents the brace 10 from migrating superiorly (i.e., in a superior direction 71 of the brace 10) up the lower leg and out of position during use. In various implementations, the first end 60 of the vertical heel stirrup strap 18 may be attached to the lateral pad outer cover 30 instead of the stabilizer body 12, the hook tab 20 may be fastened to the stabilizer body 12 instead of the lateral pad outer cover 30, and the direction in which the vertical heel stirrup strap 18 is applied may be opposite than that described above.

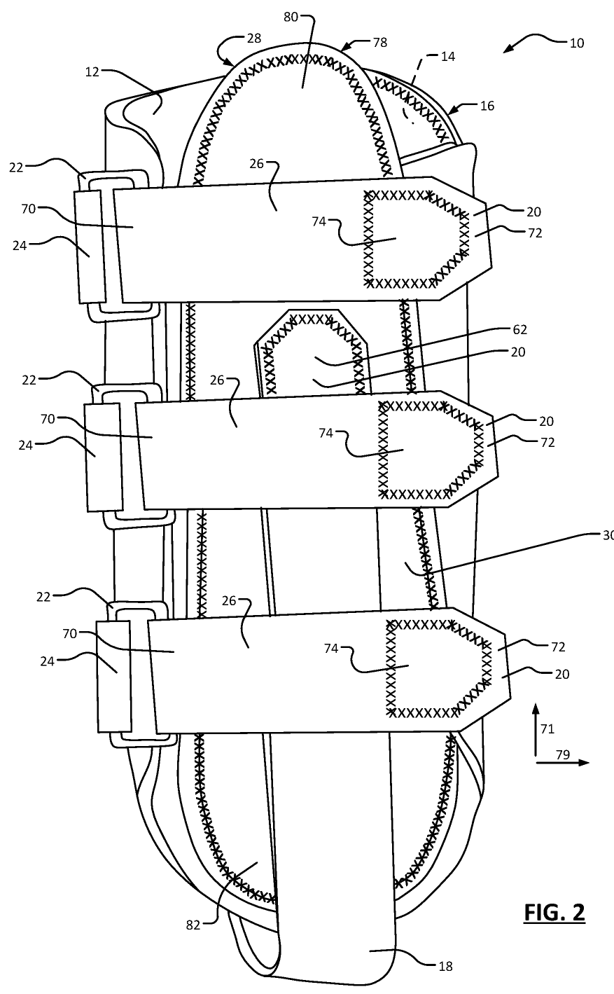

Referring now to FIGS. 2, 4, and 8, the brace 10 includes four of the hook tabs 20. Each of the hook tabs 20 can have a width W between 1 inch and 3 inches and a length L between 1 inch and 3 inches. One hook tab 20 is placed at the second end 62 of the vertical heel stirrup strap 18 as described above. The other three tabs 20 are attached at the end of the horizontal loop straps 26. Each hook tab 20 includes a plurality of hooks configured to interlock with a plurality of loops such as those found in hook-and-loop fasteners. The materials, quantity, shape, length, width, height, thickness, breathability, ability to stretch, firmness, and color of the hook tabs 20 may vary. In addition, the manner in which the hook tabs 20 are attached to the rest of the brace 10 and the direction in which the hook tabs 20 are applied may vary.

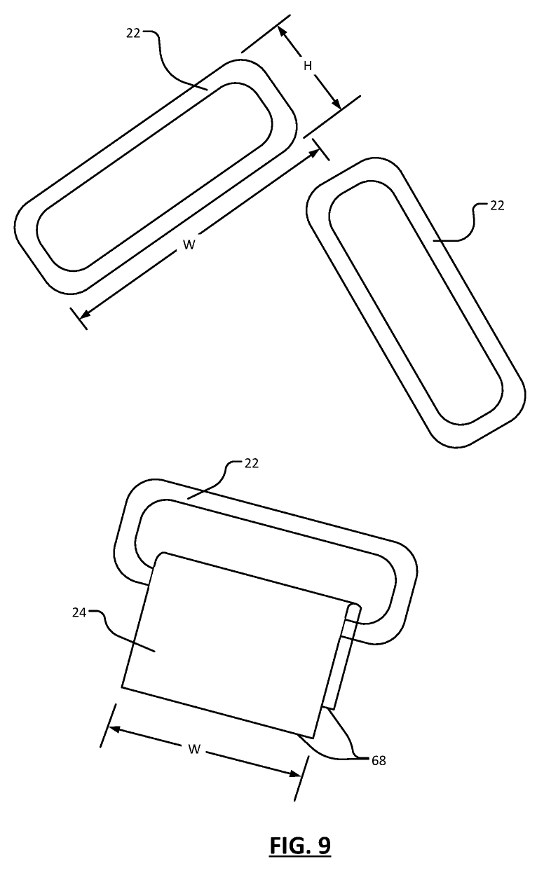

Referring now to FIGS. 2, 4, and 9, the brace 10 includes three of the loop locks 22. Each of the loop locks 22 may have a width W between 1 inch and 4 inches and a height H between 0.5 inches and 1 inch. Each loop lock 22 is sewn to the stabilizer body 12 at the posterior side of the brace 10 (i.e., at or near a lateral edge 65 of the posterior wall 39) with one loop lock 22 at the top, middle, and bottom of the brace 10 aligned with the hook securing straps 24. The horizontal loop straps 26 pass through these loop locks 22. The materials, quantity, shape, size of length, width, or height, thickness, breathability, ability to stretch, firmness, and color of the loop locks 22 may vary. In addition, the manner in which the loop locks 22 are attached to the rest of the brace 10 and the direction in which the loop locks 22 are applied may vary.

The brace 10 includes three of the hook securing straps 24. Each hook securing strap 24 has a width W between 1 inch and 3 inches and a length (i.e., distance between opposite ends 68 between 1 inch and 3 inches. Each hook securing strap 24 is wrapped around one upright of the loop locks 22 and secured (e.g., sewn) to the stabilizer body 12 at the posterior side of the brace 10 (e.g., using stitches 69) with one loop lock 22 at the top, middle, and bottom of the brace 10. The materials, quantity, shape, length, width, height, thickness, breathability, ability to stretch, firmness, and color of the hook securing straps 24 may vary. In addition, the manner in which the hook securing straps 24 are attached to the rest of the brace 10 and the direction in which the hook securing straps 24 are applied may vary.

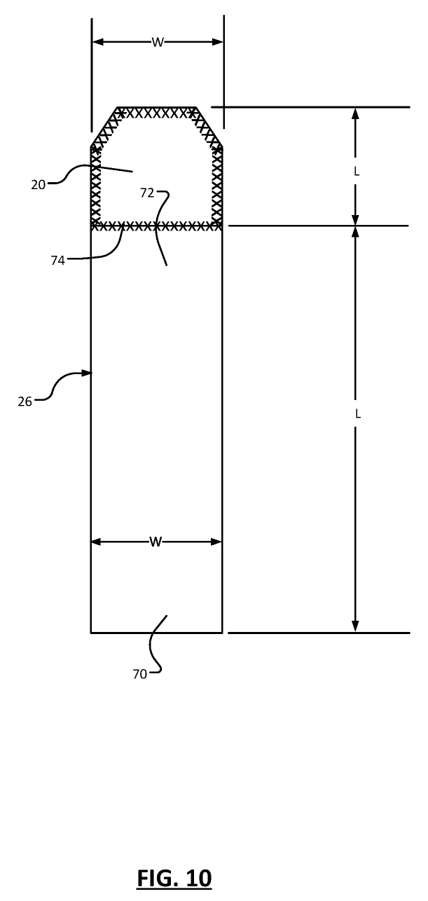

Referring now to FIGS. 2-4 and 10, each horizontal loop strap 26 can have a width W between 1 inch and 3 inches and a length L between 5 inches and 14 inches. Each horizontal loop strap 26 has a first end 70 and a second end 72 opposite of the first end 70. The first end 70 of each horizontal loop strap 26 is attached to the stabilizer body 12 at the anterior side of the brace 10 with one horizontal loop strap 26 at the top, middle, and bottom of the brace 10. The second end 72 of each horizontal loop strap 26 has a hook tab 20 attached (e.g., sewn) thereto (e.g., using stiches 74). These hook tabs 20 may be considered part of the horizontal loop straps 26. The horizontal loop straps 26 extend parallel to each other from the anterior side of the brace 10, horizontally to the lateral side of the brace 10 through the corresponding slits 76 in the lateral pad outer cover 30, to the posterior side of the brace 10 through corresponding three loop locks 22, back horizontally to the lateral side, and then to the anterior side of the brace 10. The hook tabs 20 on the horizontal loop straps 26 are then individually secured to the anterior or medial sides of the brace 10.

While applying the brace 10 to the ankle and lower leg, the athlete runs each individual horizontal loop strap 26 through its corresponding loop lock 22 at the posterior side of the brace 10. The athlete then pulls each strap 26 firmly, one at a time, from the posterior and lateral ankle and lower leg towards the anterior and medial ankle and lower leg securing each strap 26 to the anterior and medial sides of the brace 10. When applied, the three horizontal loop straps 26 act to pull the medial pad 36 of the brace 10 and a lateral pad 78 of the brace 10 together, thereby compressing the fibula and tibia together. Due to their direction of pull from posterior and lateral to anterior and medial, the horizontal loop straps 26 also act on the lateral pad 78 to pull the distal fibula in an anterior direction 79 of the brace 10 to bring the anterior distal fibula closer to the anterior distal tibia. These actions of pulling the fibula and tibia together can reduce the stress on the ligaments of the syndesmosis joint between these bones, which include the interosseous membrane, anterior inferior tibiofibular ligament, and the posterior inferior tibiofibular ligament. The position of the vertical heel stirrup strap 18 as it passes along the anterior-inferior lower leg and anterior-superior ankle can also act to limit dorsiflexion of the foot and ankle. This decrease in dorsiflexion can help prevent the foot and ankle from getting into a position susceptible for a high ankle sprain or re-aggravating a previous high ankle sprain injury. The materials, quantity, shape, length, width, height, thickness, breathability, ability to stretch, firmness, and color of the horizontal loop straps 26 can vary. In addition, the manner in which the horizontal loop straps 26 are attached to the rest of the brace 10 and the direction in which the horizontal loop straps 26 are applied may vary.

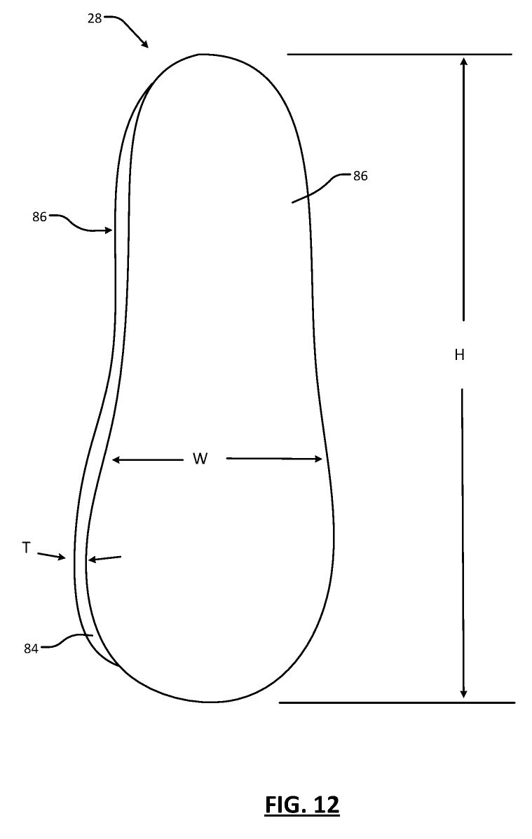

Referring now to FIGS. 2-4, 11, and 12, the lateral pad 78 of the brace 10 consists of the lateral pad inner liner 28 and the lateral pad outer cover 30. The lateral pad 78 is configured to engage the lateral side of an ankle. The lateral pad 78 may have a first width W1 adjacent to its superior end 80 and a second width W2 adjacent to its inferior end 82. The second width W2 of the lateral pad 78 may be greater than the first width W1 of the lateral pad 78. In one example, the second width W2 of the lateral pad 78 is at least 1.25 times greater than the first width W1 of the lateral pad 78. Additionally or alternatively, the first width W1 of the lateral pad 78 may be equal to the first width W1 of the medial pad 36, and the second width W2 of the lateral pad 78 may be equal to the second width W2 of the medial pad 36.

The lateral pad inner liner 28 can be teardrop-shaped as shown and can be made of a neoprene rubber 84 with a nylon laminate 86 on both sides. The lateral pad outer cover 30 can also be teardrop-shaped as shown and can be made of a neoprene rubber 88 with a hook-compatible laminate 90 on the outside and a non-stretch laminate 92 on the inside. The lateral pad inner liner 28 and the lateral pad outer cover 30 can each have a height H between 5 inches and 11 inches, a width W between 1 inch and 4 inches, and a thickness T between 2 mm and 7 mm.

The lateral pad outer cover 30 has six vertical slits 76 formed (e.g., cut) therein to allow the three corresponding horizontal loop straps 26 to pass through the lateral pad outer cover 30. These slits 76 are positioned at three levels along the lateral pad outer cover 30—top, middle, and bottom. Each level has two parallel slits 76. The slits 76 of the top and middle levels can be 0.5 to 2 inches apart. The slits 76 of the wider bottom level can be 1 to 3 inches apart.

The lateral pad inner liner 28 and lateral pad outer cover 30 are sewn together with the hook-compatible laminate 86 of the lateral pad outer cover 30 and the six vertical slits 76 on the outside and facing away from the stabilizer body 12. The lateral pad 78 is positioned with its wider end (i.e., inferior end 82) distally against the lateral malleolus of the fibula and extending proximally up the distal fibula. Each of the three horizontal loop straps 26 has one corresponding level of two slits 76 to slide through. The three horizontal loop straps 26 slide through the slits 76 of the lateral pad outer cover 30, with one horizontal loop strap 26 sliding through each level of slits 76, and secure the lateral pad 78 along the distal lateral lower leg and lateral malleolus. This acts to keep the lateral pad 78 at the same horizontal level as the medial pad 36, which allows the horizontal loop straps 26 to pull the lateral pad 78 into the distal fibula to both compress the distal fibula toward the distal tibia and pull the distal fibula anteriorly (i.e., in the anterior direction 79) and medially (i.e., in a medial direction 93 of the brace 10) towards the anterior distal tibia. The materials, quantity, shape, length, width, height, thickness, breathability, ability to stretch, firmness, and color of the lateral pad inner liner 28 and the lateral pad outer cover 30, and the number of slits 76 in the lateral pad outer cover 30, may vary. In addition, the manner in which the lateral pad inner liner 28 and lateral pad outer cover 30 are attached to the stabilizer body 12 may vary. Further, the lateral pad inner liner 28 and the lateral pad outer cover 30 may or may not be attached to the stabilizer body 12.

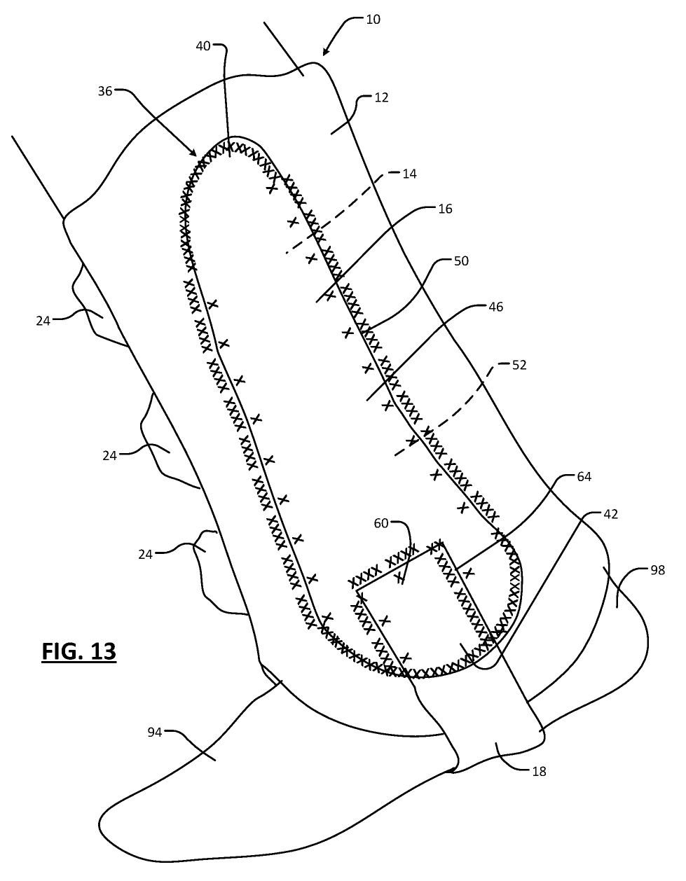

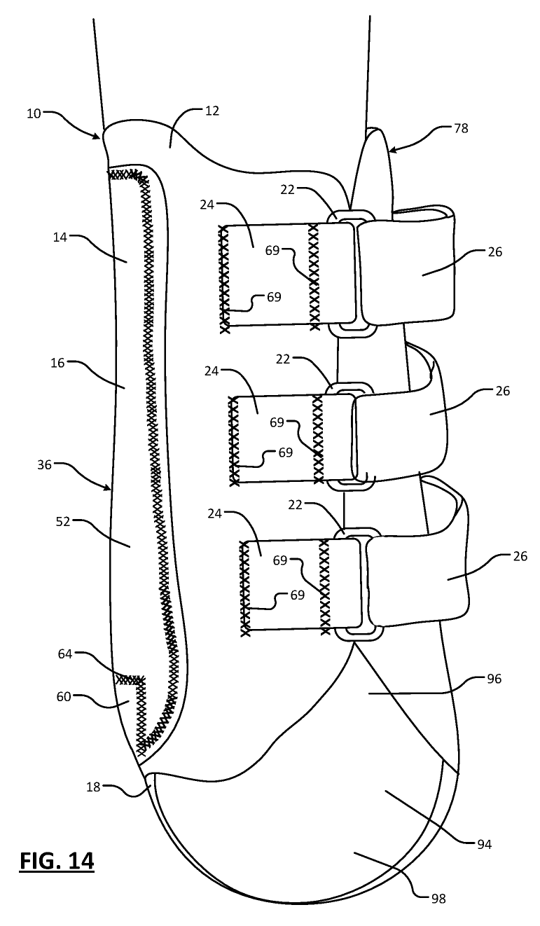

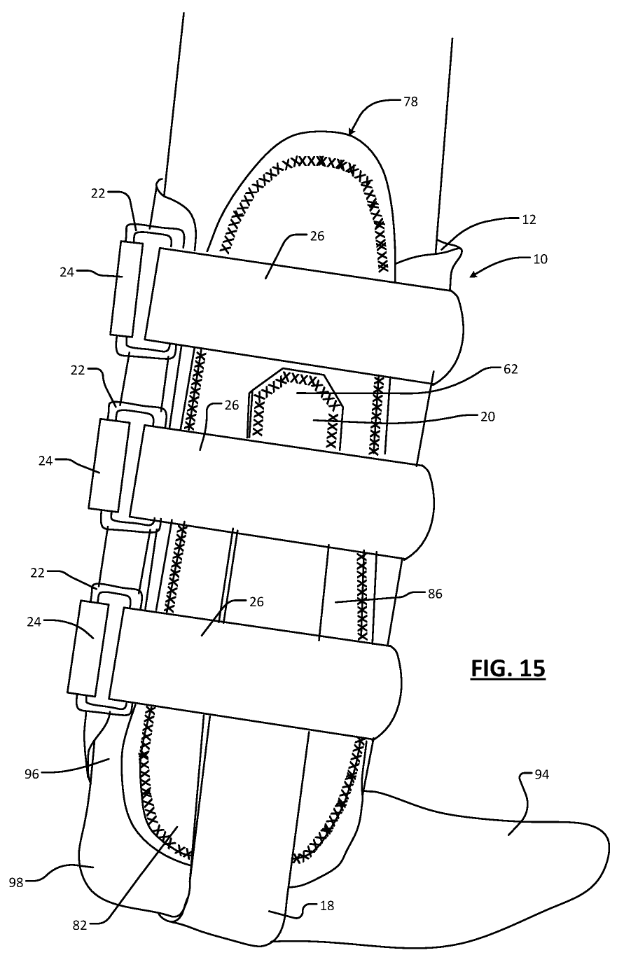

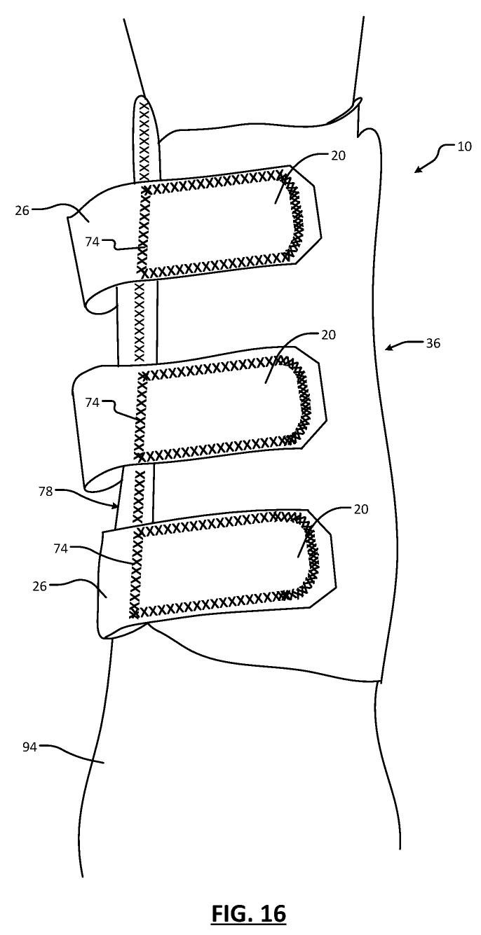

Referring now to FIGS. 13-16, an example method of securing the brace 10 to a foot 94 and ankle 96 will now be described. Position the foot 94 and ankle 96 in a neutral position. Open the brace 10 with the vertical heel stirrup strap 18 and three horizontal loop straps 26 all detached. Position the brace 10 so that the wider portions of the medial and lateral pads 36 and 78 are inferior and the narrower portions are superior. Apply the brace 10 in this position to the anterior ankle and lower leg so as the medial pad 36 is placed against the distal tibia of the medial ankle and lower leg and the lateral pad 78 is placed against the distal fibula of the lateral ankle and lower leg.

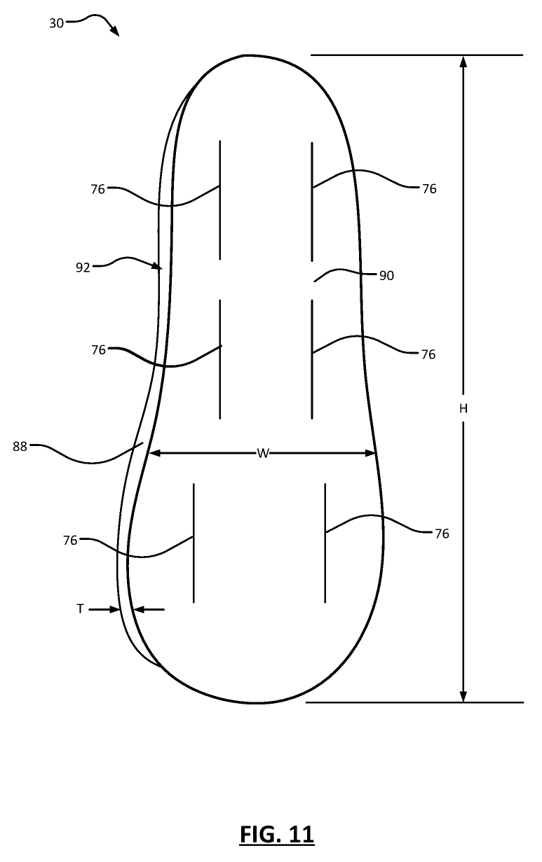

Next, apply the vertical heel stirrup strap 18 by pulling the strap 18 under a heel 98 and then up along the lateral ankle and lower leg. Apply moderate tension to the strap 18 and secure it, using the hook tab 20, to the hook-compatible laminate 86 of the lateral pad 78 along its vertical midline between the six vertical slits 76 (FIG. 11). Then, position the foot 94 and ankle 96 into slight plantarflexion. This positioning allows the talus to shift forward out of the mortis of the ankle 96 so the tibia and fibula can be effectively compressed together without resistance or blockage from the talus.

Then, apply the three horizontal loop straps 26, one at a time, through each of their corresponding posterior loop locks 22. Once through the loop locks 22, pull the three horizontal loop straps 26 back towards the lateral side, then around to the anterior and medial sides of the brace 10. Apply firm tension, as much as tolerable, to each individual strap and secure them, using the hook tabs 20, to the hook-compatible anterior and medial surfaces of the brace 10. Loosen and re-apply each individual horizontal loop strap 26 as needed to adjust the position and tension of each strap 26. This adjusts the forces on the ankle 96 and lower leg and limit dorsiflexion of the foot 94 and ankle 96.

Again, the materials, quantities, shape, length, width, height, thickness, breathability, ability to stretch, firmness, and color of the brace 10 and its individual components may vary. In addition, the direction in which the components of the brace 10 are applied may vary, and how the components of the brace 10 are attached to the rest of the brace 10 may vary. In an example of the latter, whether the components of the brace 10 are joined together using a hook-and-loop fastener, sewing, glue, rivets, or other means of joining may be varied. Further, one or more of the components of the brace 10 may be omitted. Moreover, the position of the athlete's foot, ankle, and lower leg during application of the brace 10 may all vary.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

What is claimed is:

1. An ankle brace comprising:
a medial pad configured to engage a medial side of an ankle;
a lateral pad configured to engage a lateral side of the ankle;
a posterior wall attached to the medial pad and configured to engage a posterior side of the ankle; and
at least one horizontal strap configured to be secured to the lateral pad, secured to the posterior wall, and pulled in an anterior direction to apply an anterior force to a distal fibula of the ankle that brings the distal fibula closer to a distal tibia of the ankle and thereby decreases stress on ligaments connecting the distal fibula and the distal tibia to one another.

2. The ankle brace of claim 1 further comprising at least one ring attached to a lateral edge of the posterior wall, wherein the at least one horizontal strap is configured to be inserted through the at least one ring to secure the at least one horizontal strap to the posterior wall.

3. The ankle brace of claim 2 further comprising an anterior wall attached to the medial pad and configured to engage an anterior side of the ankle, wherein the at least one horizontal strap is attached to the anterior wall and is configured to be inserted through slits in the lateral pad, through the at least one ring, and fastened to the anterior wall to secure the at least one horizontal strap to the lateral pad.

4. The ankle brace of claim 3 wherein the at least one horizontal strap includes a first portion attached to the anterior wall and having a plurality of loops, and a second portion having a plurality of hooks configured to engage the plurality of loops and thereby fasten the at least one horizontal strap to the anterior wall.

5. The ankle brace of claim 3 wherein the lateral pad includes an inner liner and an outer cover that is attached to the inner liner, wherein the outer cover defines the slits in the lateral pad.

6. The ankle brace of claim 3 wherein the anterior and posterior walls are formed by a flexible sheet, the flexible sheet including a medial portion that forms an inner liner of the medial pad.

7. The ankle brace of claim 6 wherein the medial pad includes the medial portion of the flexible sheet, a medial insert, and an outer cover attached to the medial portion of the flexible sheet to form a pocket that encloses the medial insert.

8. An ankle brace comprising:
a medial pad configured to engage a medial side of an ankle;
a lateral pad configured to engage a lateral side of the ankle;
a vertical strap attached to one of the medial and lateral pads and configured to be wrapped around a heel and secured to the other one of the medial and lateral pads to prevent the ankle brace from moving in a superior direction; and
at least one horizontal strap securing the medial and lateral pads to one another and configured to be pulled in at least one of a medial direction and a lateral direction to move the medial and lateral pads toward one another such that the medial and lateral pads apply a compression force to the ankle in the medial and lateral directions that urges a distal fibula of the ankle and a distal tibia of the ankle toward one another, wherein the vertical strap is flexible so that the vertical strap does not counteract the compression force applied by the medial and lateral pads.

9. The ankle brace of claim 8 wherein the vertical strap is attached to the medial pad and configured to be wrapped around the heel and secured to the lateral pad.

10. The ankle brace of claim 8 wherein the lateral pad includes a plurality of loops and the vertical strap includes a plurality of hooks configured to engage the plurality of loops to secure the vertical strap to the lateral pad.

11. The ankle brace of claim 8 wherein the at least one horizontal strap is configured to be pulled in the lateral direction to apply the compression force to the ankle.

12. The ankle brace of claim 8 further comprising:
a posterior wall attached to the medial pad and configured to engage a posterior side of the ankle; and
at least one ring attached to a lateral edge of the posterior wall, wherein the at least one horizontal strap is configured to be inserted through the at least one ring to secure the at least one horizontal strap to the posterior wall.

13. The ankle brace of claim 8 wherein at least a portion of the vertical strap is made of neoprene rubber having a thickness between 2 millimeters and 7 millimeters.

14. The ankle brace of claim 8 wherein the medial pad has a first stiffness and the lateral pad has a second stiffness that is greater than the first stiffness.

15. An ankle brace comprising:
a medial pad configured to engage a medial side of an ankle, the medial pad having a first width adjacent to its superior end and a second width adjacent to its inferior end that is greater than the first width;
a lateral pad configured to engage a lateral side of the ankle, the lateral pad having a third width adjacent to its superior end and a fourth width adjacent to its inferior end that is greater than the first width; and
at least one horizontal strap securing the medial and lateral pads to one another and configured to be pulled in at least one of a medial direction and a lateral direction to move the medial and lateral pads toward one another such that the medial and lateral pads apply a compression force to the ankle in the medial and lateral directions that urges a distal fibula of the ankle and a distal tibia of the ankle toward one another.

16. The ankle brace of claim 15 wherein the second width is at least 1.25 times greater than the first width, and the fourth width is at least 1.25 times greater than the third width.

17. The ankle brace of claim 15 wherein the lateral pad includes an inner liner and an outer cover that is attached to the inner liner.

18. The ankle brace of claim 17 wherein each of the inner liner and the outer cover is made from neoprene rubber having a thickness between 2 millimeters and 7 millimeters.

19. The ankle brace of claim 15 wherein the medial pad includes an inner liner, a medial insert, and an outer cover attached to the inner liner to form a pocket that encloses the medial insert.

20. The ankle brace of claim 19 wherein each of the inner liner and the outer cover is made from neoprene rubber having a thickness between 2 millimeters and 7 millimeters, and the medial insert is made from high-density foam having a thickness between 2 millimeters and 10 millimeters.
